(19) United States
(12) Plant Patent Application Publication
(10) Pub. No.: US 2013/0081160 P1
(43) Pub. Date: Mar. 28, 2013

(54) Columnar parrotia persica named JL columnar

(50) ;

(71) Applicant: John Lewis, Salem, OR (US)

(72) Inventor: John Lewis, Salem, OR (US)

(21) Appl. No.: 13/200,724

(22) Filed: Sep. 28, 2011

(51) Int. Cl. A01H 5/00

(52) U.S. Cl. PLT/216

(57) ABSTRACT

A new cultivar of Ornamental Parrotia Persica plant named JLColumnar that is characterized by a columnar growth habit, leaves which are solid purple when they break dormancy in the spring, turn to green with purple halo margins, and display a two phase fall color. The leaves are slightly smaller than in the genus.

### GENUS

Parrotia

### SPECIES

Persica

### VARIETAL DENOMINATION

JLColumnar

### BACKGROUND OF THE INVENTION

The new Parrotia persica tree was discovered as a seedling of unknown parentage while growing in a cultivated area tended by man.

Plants of the new variety have been asexually reproduced at Salem, Oreg., U.S.A., through the use of softwood cuttings. The characteristics of the new variety have been found to be strictly transmissible by such asexual propagation, and the new variety reliably reproduces in a true-to-type manner from one generation to another.

### BRIEF SUMMARY OF THE INVENTION

The subject variety exhibits the following combination of characteristics:

(a) Displays a columnar habit

(b) Leafs break dormancy in the spring with solid purple leaves

(c) New leaves transform to green with purple halo margins

(d) Displays two phase fall color

(c) Forms slightly smaller leaves

The variety can be readily distinguished from previously known Parrotia persica varieties in view of the recited combination of characteristics. The growth habit is columnar in form and differs significantly from the species which is rounded to broad-rounded by a horizontal branching appearance. The shape is similar to no other cultivars. Additionally, it forms leaves which are slightly smaller in length and width.

The variety meets the needs of the horticultural industry and is well suited for growing as attractive distinctive ornamentation in the landscape.

### BRIEF DESCRIPTION OF THE PHOTOGRAPHS

FIG. 1 illustrates a close-up view of a 4 year old specimen of the new variety, growing on its own root.

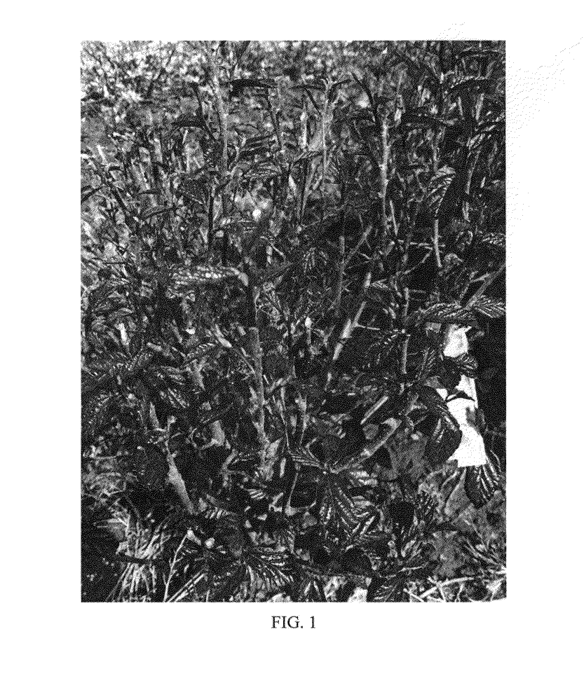

FIG. 2 illustrates the same 4 year old, own root specimen, up close to show the emerging purple leaves of the new variety.

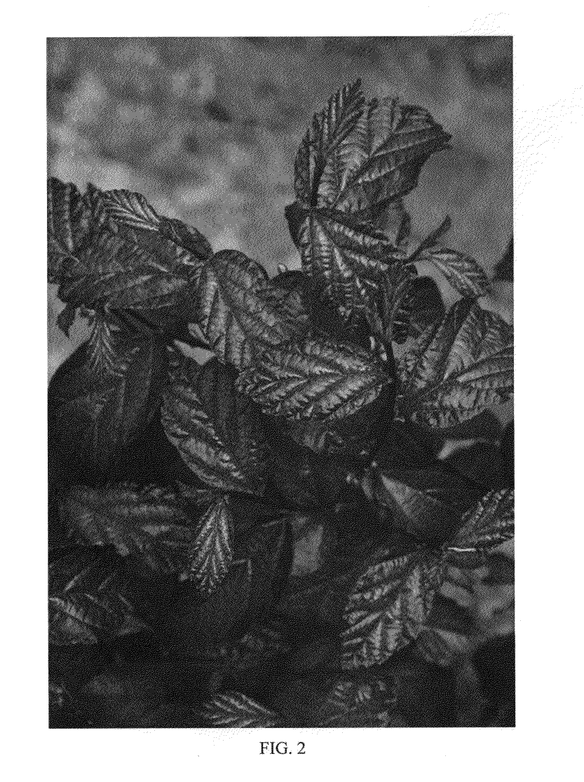

FIG. 3 illustrates the leaves transform to green with purple halo margins.

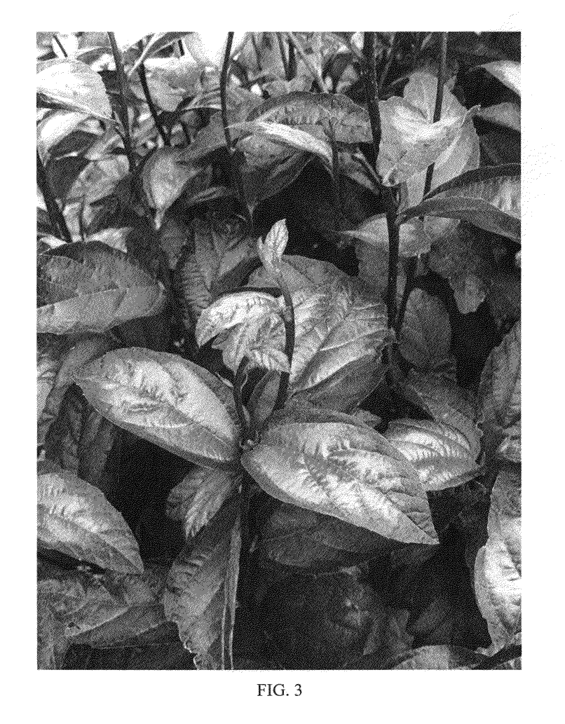

FIG. 4 illustrates the columnar growth habit of the new variety.

FIG. 5 illustrates the first phase of the fall color change.

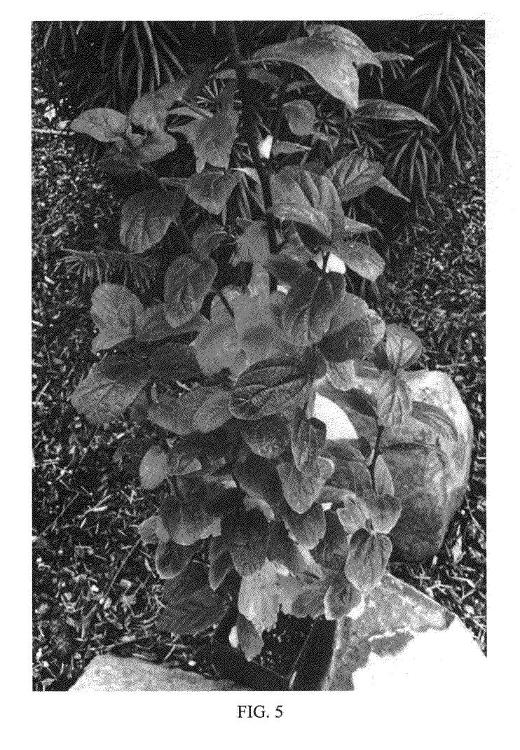

FIG. 6 illustrates the second phase of the fall color change.

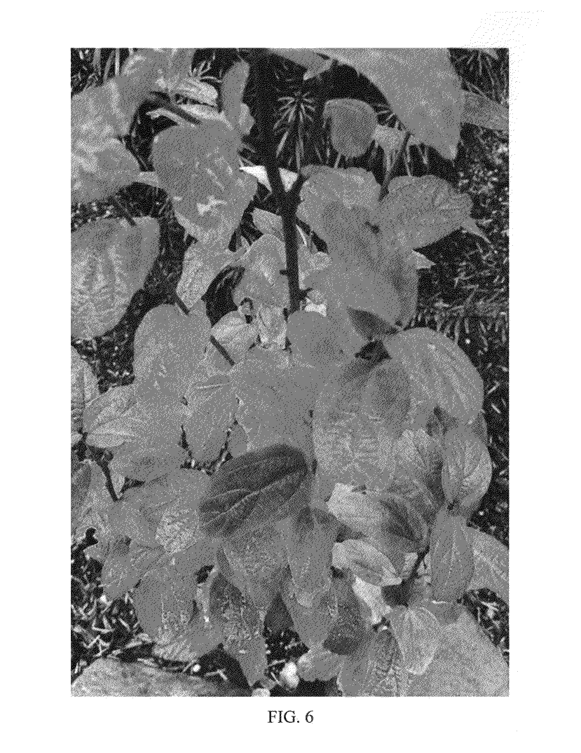

FIG. 7 illustrates the smaller leaf size compared to the larger leaf of the species.

### DETAILED BOTANICAL DESCRIPTION OF PREFERRED EMBODIMENT

The chart used in the identification of color is the R.H.S. Colour Chart of The Royal Horticultural Society, London, England. Other reference to color is to be accorded its ordinary dictionary significance. The description is based upon the observation of plants of the new variety having an age of approximately 4 years while being grown outdoors at Salem, Oreg., U.S.A.

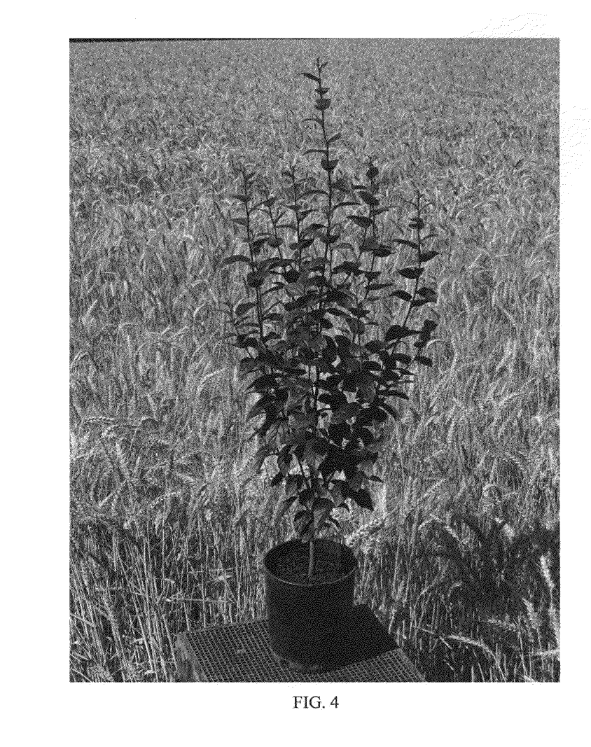

The new variety exhibits an extremely columnar growth habit with a fast growth rate as shown in FIG. 4 in this regard.

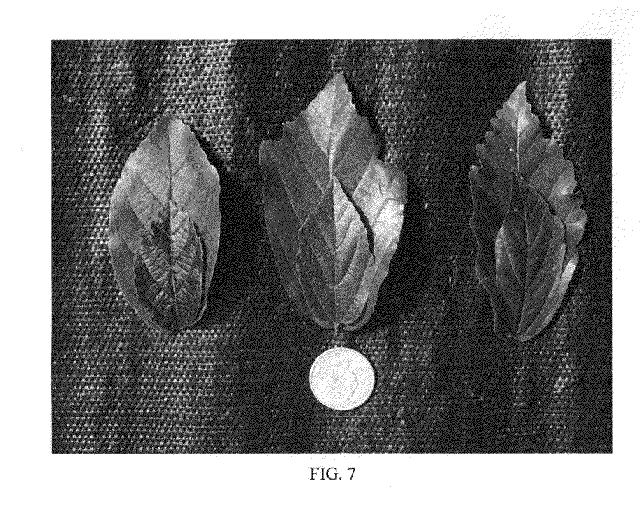

The vegetative parts of the new variety are typical of the genus except as specifically indicated hereafter. The alternate, simple oval to obovate-oblong leaves are lanceolate caduceus stipules, obtuse, rounded to sub-cordate at base, coarsely crenate-dentate about the middle, glabrous above with sparse pubescence below, undulate, lustrous. See FIG. 7 in this regard. The petiole is approximately ⅛″ in length which is typical of the species. The species leaves are normally 2½″ to 5″ long and 1″ to 2½″ wide. The new variety has leaves approximately 2½ to 3½″” in length and around 1½″ to 2″ in width. Compared to standard Parrotia Persica, its leaves are much smaller, and narrow.

The emerging leaves of the new variety in the spring on the upper surface are like Red Purple Group 71A. As new spring growth emerges and leaves form to full size, leaves transition to Greyed-Purple 187 A-D. As leaves mature, they deepen to Green Group 141A with a haloed margin of Red-Purple Group 74A. Mature leaves are solid Color Group 141A

Immature twigs, mature branches and bark are all typical in color to that of the species and do not differ significantly or increase the ornamental value of the tree over other varieties.

The blossom appearance of the new variety is similar to that of the genus. The new variety forms inconspicuous blooms which are perfect, apetalous. Crimson maroon flowers with crimson stamens ⅓″ across with yellow anthers. Buds generally opening to Red-Purple Group 57 B

The overall configuration of the flowers is generally typical of the genus. The flowers are perfect, apetalous and sparse. Blossoming commonly occurs during late march at Salem, Oreg., U.S.A., where it commonly extends over approximately 1-2 weeks depending upon the weather conditions that are encountered. The flowers are commonly borne attached to stem. Each stem has multiple flowers which are not fragrant. The reproductive organs are typical of a Parrotia persica and other species and cultivars of angiosperm flowering plants.

During observations to date, fruit has not been formed and is not considered to be ornamental in nature.

When grown at Salem, Oreg., U.S.A., the new variety has exhibited disease resistance under observations to date and has not been affected to any degree by common Parrotia diseases or by insects which commonly attack Parrotia. The new variety has proven to be hardy when tested in U.S.D.A. Hardiness Zone 5.

The terms and expressions which have been employed in the foregoing specification are used therein as terms of description and not of limitation, and there is no intention, in the use of such terms and expressions, of excluding equivalents of the features shown and described or portions thereof, it being recognized that the scope of the invention is defined and limited only by the claims which follow.

What is claimed is:

1. A new and distinct cultivar of Ornamental Parrotia Persica named JL Columnar as described and illustrated herein.
